(19) United States
(12) Patent Application Publication
(10) Pub. No.: US 2005/0170014 A1
(43) Pub. Date: Aug. 4, 2005

(54) Carbonyl iron pharmaceutical dosage forms for the treatment of iron-deficiency anemia

(76) Inventors: Anandi Krishnan, Mumbai (IN); M. E. Kannan, Mumbai (IN); Preethi B. Rao, Mumbai (IN)

Correspondence Address: KENYON & KENYON, ONE BROADWAY, NEW YORK, NY, 10004, US

(21) Appl. No.: 10/772,745

(22) Filed: Feb. 4, 2004

(30) Foreign Application Priority Data
Jan. 30, 2004 (IN) 106/MUM/2004

(51) Int. Cl. A61K 33/26; A61K 31/525; A61K 31/714

(52) U.S. Cl. 424/646; 514/52; 514/251; 424/451

(57) ABSTRACT

The present invention relates to a pharmaceutical composition including an active pharmaceutical ingredient for the treatment of iron-deficiency anemia and related disorders. The active pharmaceutical ingredient is preferably carbonyl iron. The formulation of the pharmaceutical composition of the present invention ensures enhanced absorption of iron in its ferrous form. Also contemplated by the present invention are the methods of making the pharmaceutical composition.

### CLAIM FOR PRIORITY

This application claims priority from Indian provisional application number 106/MUM/2004 filed Jan. 30, 2004. The priority application is incorporated by reference herein in its entirety.

### FIELD OF THE INVENTION

The present invention relates to carbonyl iron pharmaceutical dosage forms for the treatment of iron-deficiency anemia and related disorders. The pharmaceutical formulations of the present invention provide enhanced absorption of iron in its ferrous form.

### BACKGROUND OF THE INVENTION

Anemia is a condition in which the numbers of red blood cells in a patient are less than normal, resulting in an insufficient amount of oxygen in tissues and organs. Iron deficiency is the most common cause of anemia in the world and is characterized by patients exhibiting low serum iron, increased serum iron-binding capacity, decreased serum ferritin, and decreased marrow iron stores. A patient suffering from iron deficiency anemia may exhibit pallor of the skin and mucous membranes, shortness of breath, palpitations of the heart, lethargy, and fatigability. Moreover, patients suffering from iron deficiencies exhibit a feedback mechanism that enhances iron absorption. In turn, the body regulates iron stores by absorption.

Regardless of its original form, when taken orally, iron is oxidized into its ferric state. The ferric form of iron is typically insoluble. In addition, its precipitation may be prevented by gastric acidity or solubilizing agents (e.g. ascorbate). Iron is absorbed by intestinal mucosal cells in the duodenum and upper jejunum. Once absorbed, iron is coupled to transferrin which is present in the blood stream, and delivered to the cells of the body.

The standard therapy for iron deficiency anemia has been the administration of ferrous salts. Iron absorption from these salts occurs predominantly in the duodenum and upper jejunum. Ionic iron salts include ferrous sulphate and ferric ammonium citrate. The use of ferrous salts, however, has many disadvantages, such as iron overload and iron toxicity. In addition, the gastrointestinal side effects, such as abdominal pain, nausea, vomiting, diarrhea and constipation, may be severe. An alternative treatment has been the administration of elemental, uncharged iron powder which is nontoxic compared to the ferrous salts. Elemental iron preparations that are produced by a hydrogen-reduction process have poor bioavailability when compared to ferrous salts. The poor bioavailability of elemental iron is due primarily to its relatively large particle size (i.e. about 50 μm) and its relatively limited reactive surface area.

More recently, an elemental iron preparation known as “carbonyl iron” powder has been introduced. The “carbonyl” part of the name does not refer to the composition of the iron particles, but rather to the manufacturing process. In the manufacturing process, there is controlled heating of vaporized iron pentacarbonyl, which leads to the deposition of uncharged, elemental iron as submicroscopic crystals. These crystals form microscopic spheres of less than 5 μm in diameter. The preparation is more than 98% pure. As a food additive, carbonyl iron has been shown both in experimental animals and in humans to be well absorbed and utilized for hemoglobin synthesis.

In addition to its smaller particle size, the gastrointestinal side-effects of carbonyl iron are less severe than conventional iron salts. Carbonyl iron is also less likely to cause iron overload and toxicity. Further, the smaller particle size and the need for an acidic environment (e.g. hydrochloric acid (pH <2)) for solubilization give advantages to carbonyl iron. Within the stomach, carbonyl iron is oxidized to the ferrous form. Hydrochloric acid provides the hydrogen ion required for oxidation. In vitro studies show that little or no carbonyl iron is solubilized or absorbed unless the pH is less than 2. Similarly, gut loop studies show that little or no carbonyl iron is absorbed in vivo unless it is exposed to a pH of less than 2. In contrast, appreciable amounts of both ferrous and ferric iron can be absorbed at a pH of 3, and ferrous iron can be absorbed up to a pH of 6. The gastric pH increases to 6 with larger doses of carbonyl iron which indicates that protons are consumed in the conversion of particulate iron to soluble ionized iron. It also appears that the amount of ionized iron produced is limited by the rate of acid secretion of the gastric mucosa. Thus, the requirement for ionization and solubilization account for the safety of carbonyl iron. After conversion to the ferrous form in the stomach, carbonyl iron is indistinguishable from that of iron ingested as ferrous sulphate.

There have been attempts to provide an iron supplement in a controlled release dosage form. For example, U.S. Pat. No. 6,521,247 to devries, discloses a controlled release iron supplement solid dosage form comprising a first composition for slowly delivering a pharmaceutically acceptable iron compound (carbonyl iron) and a second composition for rapidly delivering a pharmaceutically acceptable iron compound (ferrous sulphate). Because the carbonyl iron needs a lower pH environment to get converted into the absorbable ferrous form, the time period available for the immediate release dosage form to reside in the stomach is very short. Hence, the majority of the iron remains unconverted to the absorbable, ferrous form and is excreted.

Accordingly, there remains a need for a carbonyl iron pharmaceutical dosage form that will provide substantially complete absorption of the solubilized iron over a prolonged period of time, an improved side effect profile and improved patient compliance.

### SUMMARY OF THE INVENTION

The present invention relates to carbonyl iron pharmaceutical dosage forms for the treatment of iron-deficiency anemia and related disorders. The pharmaceutical formulations of the present invention provide enhanced absorption of iron in its ferrous form.

The pharmaceutical formulation of the present invention may be a tablet of carbonyl iron. Carbonyl iron is adsorbed over directly compressible grade of sorbitol. The adsorption potential of sorbitol is very high rendering a uniform blend of drug and other excipients. The viscolyzing agent may be low molecular weight polyethylene oxides. The swelling agent in the pharmaceutical composition may be cross-linked polyvinylpyrrolidone, which swells several times its original volume and entraps the gas that is generated with the help of a gas generating agent, making the formulation buoyant. The mucoadhesive agent incorporated in the formulation may be polyacrylic acid polymer. Gas generation in the formulation is by the hydrogen gas which is released when carbonyl iron reacts with hydrochloric acid in the stomach to form the absorbable ferrous form. Synergistically, gas generation is also due to the incorporation of sodium bicarbonate, which liberates hydrogen and carbon dioxide gas in contact with hydrochloric acid of the stomach.

The dosage form of the invention is intended to be administered to the patient after meals during bed time. Once administered the tablet swells and the gas released from the formulation makes the dosage form buoyant. The buoyant dosage form of the invention resides in the stomach and retains its structural integrity for a prolonged duration of time of about 4 to 6 hours. During the course of time, the formulation starts to disintegrate slowly thereby facilitating the conversion of carbonyl iron present in the discrete mass to the absorbable ferrous form. The mucoadhesive agent in the dosage form, further contributes to the conversion to the absorbable form facilitating the contact between the disintegrated mass and the mucus membrane, where the hydrochloric acid secretion is maximal.

One aspect of the present invention is to provide an oral pharmaceutical composition of carbonyl iron that generates and entraps a gas in a matrix upon contact with gastric fluids.

Another aspect of the present invention is to provide an oral pharmaceutical composition of carbonyl iron that provides increased gastric residence, and thereby, a longer period of residence of the drug delivery system in the upper part of the gastro intestinal system.

Another aspect of the present invention is to provide an oral pharmaceutical composition of carbonyl iron that delivers the drug at a controlled rate such that the drug is delivered over a period of time which is same as or less than the period of residence of the delivery system in the absorptive regions of the gastrointestinal tract.

Another aspect of the present invention is to provide an oral pharmaceutical composition of carbonyl iron that provides enhanced absorption of iron in its ferrous form by remaining in the stomach for a longer time, as compared to other controlled release compositions of carbonyl iron.

### DEFINITIONS

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

As used herein the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ f127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in Howard C. Ansel et al., Pharmaceutical Dosage Forms and Drug Delivery Systems, (7th Ed. 1999); and Alfonso R. Gennaro et al., Remington: The Science and Practice of Pharmacy, (20th Ed. 2000), which are both incorporated by reference herein.

### DETAILED DESCRIPTION OF THE INVENTION

The present invention provides a pharmaceutical composition in the form of an orally administered dosage form of an active pharmaceutical ingredient. The dosage form of the present invention is designed to reside at a particular portion of the patient's gastrointestinal tract and effectively deliver the active pharmaceutical ingredient from the dosage form to a patient over a specific period of time. The dosage form avoids dose dumping and results in a therapeutic administration of the active pharmaceutical ingredient to a person with iron deficiency anemia and related disorders.

According to the present invention, the pharmaceutical composition is suitable for controlled delivery of an active pharmaceutical ingredient that is absorbed only from the upper parts of the gastrointestinal tract with a specific absorption window.

The pharmaceutical composition of the present invention comprises one or more active pharmaceutical ingredient or neutraceutical, a carrier, a swelling agent, a viscolyzing agent, a gas generating agent, a mucoadhesive agent and other pharmaceutically acceptable additives. The dosage form of the present invention forms a porous matrix, the characteristics of which can be modified by altering the ratios and amounts of the above mentioned components. The composition can therefore be designed to obtain the optimal rate of release of the active pharmaceutical ingredient or neutraceutical from the dosage form depending upon the requirement of the respective active pharmaceutical ingredient. The various components of the pharmaceutical composition are described in more detail.

The preferred active pharmaceutical ingredient (API) of the present invention is carbonyl iron. Carbonyl iron is oxidized to the ferrous form within the stomach. The hydrogen ion required for oxidation is derived from hydrochloric acid present in the gastric environment of the stomach. A pH of less than 2 is required for ionization and solubilization of carbonyl iron to its absorbable ferrous form. The absorption of ferrous iron takes place in the duodenum. Therefore, it becomes necessary that the formulation stay in the stomach for longer periods of time sufficient to enable the conversion of the maximum amount of iron in the formulation to its absorbable ferrous form.

A combination of drugs that are typically administered together may be the active pharmaceutical ingredients of the pharmaceutical composition of the present invention. In the present invention, Vitamin B12 and Folic acid are included in the formulation with carbonyl iron because they also are used to treat iron-deficiency anemia and related disorders. The amount of active pharmaceutical ingredients in the composition generally varies from about 1% to about 15% by weight of the composition. Preferably, the amount of active pharmaceutical ingredients varies from about 2% to about 10% by weight of the composition.

The pharmaceutical formulation of the present invention includes a carrier. The carrier may be sorbitol. Preferably, Sorbitol Instant, available from E.Merck, is used in the composition as a carrier. The combination of carbonyl iron and sorbitol may be successfully tabletted because sorbitol has an extremely high adsorption potential for carbonyl iron. It also has the ability to retain adsorption without carbonyl iron segregating from the sorbitol carrier. This occurs despite the significant density differences between the sorbitol carrier and carbonyl iron. Sorbitol also has properties of a diluent. The amount of carrier generally varies from about 10% to about 90% by weight of the composition.

The pharmaceutical composition of the present invention contains a viscolyzing agent. As used herein, the term viscolyzing agent is intended to mean a compound used to impart increased viscosity in a dosage form. Such compounds include, by way of example and without limitation, cellulose derivatives, clays/minerals, natural gums, synthetic gums, pyrrolidone derivatives, poly vinyl alcohols, polyethylene oxides, vinyl acetate/crotonic acid copolymers, maleic anhydride/methyl vinyl ether copolymers, their derivatives, combinations thereof and other such materials known to those of ordinary skill in the art. The viscolyzing agent may be polyethylene oxides (PEO). Polyethylene oxide is a non-ionic homopolymer of oxyethylene groups (about 2000 to over 100,000) that are water soluble. They are thermoplastic agents that are readily calendared, extruded, injection molded or cast. Polyethylene oxides provide extended release via the hydrophilic matrix approach. Polyethylene oxides on exposure to water or gastric juices hydrate and swell rapidly to form hydrogels with properties ideally suited for controlled release formulations. Polyethylene oxides are non-ionic, so no interaction between the drug and the polymer is to be expected. Polyethylene oxides are proven to be excellent release retarding and mucoadhesive polymers. Polyethylene oxides are commercially available in various grades, under several trade names including Polyox® WSR N-10, WSR N-80, WSR N-750, WSR-705, WSR1105, WSR N-12K, WSR N-60K, WSR-301, WSR coagulant and WSR-303, available from Dow of Midland, Mich. The different grades under the given trade name represent the differences in oxyethylene content, as well as molecular weight. The pharmaceutical composition of the present invention may contain one polyethylene oxide grade alone or a combination of different grades of polyethylene oxides. The amount of viscolyzing agent in the composition generally varies from about 0.1% to about 20% by weight of the composition. Preferably, the amount of viscolyzing agent varies from about 0.25% to about 15% by weight of the composition.

According to the present invention, the pharmaceutical composition contains a swelling agent which is capable of swelling to greater than its original volume, when coming into contact with an aqueous fluid, such as a gastrointestinal fluid. It is preferable that the swelling agent is capable of swelling to at least twice its original volume. Examples of the swelling agents that may be used in the present invention include sodium carboxymethylcellulose, calcium carboxymethylcellulose, cross-linked polyvinylpyrrolidone, cross-linked carboxymethylcellulose sodium, and sodium starch glycolate. The swelling agents may be compounds that belong to the class of compounds known as superdisintegrants, which usually function to promote disintegration of a tablet by absorbing large amounts of water and thereby swelling. This expansion, as well as the hydrostatic pressure, causes the tablet to burst. In a tablet which also contains a gas generating component, the tablet is expected to disintegrate instantly upon contact with aqueous fluid. Remarkably, it has been found that in the presence of an instantly acting viscolyzing agent, the generated gas is entrapped and the superdisintegrants act as a swelling agent. The combination of the gas generating agent, swelling agent, and viscolyzing agent act as a modified release drug matrix. Preferably, the swelling agent is cross-linked polyvinyl pyrrolidone. The swelling agent, which normally swells to several times its original volume in water, exhibits a controlled swelling in the presence of the viscolyzing agent. The amount of swelling agent in the composition generally varies from about 1% to about 25% by weight of the composition. Preferably, the amount of swelling agent varies from about 5% to about 15% by weight of the composition.

The gas generating agent may consist of a single substance known to produce gas upon contact with gastric fluid, or may consist of a gas generating couple. Examples of the gas generating agent that may be used in the present invention include carbonates, for example, calcium carbonate or sodium glycine carbonate, bicarbonates, for example, sodium hydrogen carbonate or potassium hydrogen carbonate, and sulfites, for example, sodium sulfite, sodium bisulfite, or sodium metabisulfite.

The gas generating agent interacts with an acid source triggered by contact with water or simply with gastric fluid to generate carbon dioxide or sulfur dioxide. The carbon dioxide or sulfur dioxide gets entrapped within the hydrated gel matrix of the swelling composition. The gas generating agent may be carbonates and bicarbonates that are present in the amounts from about 0.1% to about 20% by weight of the composition. Preferably, the gas generating agent is present in an amount from about 0.25% to about 10% by weight of the composition. In the present invention, the carbonyl iron itself generates hydrogen gas on contact with the hydrochloric acid in the stomach, according to the following equation:
Fe^{o+}+2 HCl→Fe²⁺Cl₂²⁻+H_{2↑}

Sodium bicarbonate and carbonyl iron synergistically act as gas generating agents in the formulation. The generated gas gets entrapped in the swollen gel matrix of the tablet making it float and thereafter slowly disintegrates.

According to the present invention, the pharmaceutical composition contains a mucoadhesive agent, which may be a cross-linked polyacrylic acid. The cross-linked polyacrylic acid may be a carbomer, available under the brand name Carbopol® from B. F. Goodrich of New York, N.Y. These are synthetic high molecular weight polymers of acrylic acid that are cross-linked with either allyl sucrose or allyl ethers of pentaerythritol. They contain between about 56% and about 68% of carboxylic acid (—COOH) groups as calculated on the dry basis. A number of different carbomer grades are commercially available that vary in their molecular weight, degree of cross-linking, polymer structure and residual components. These differences account for the specific rheological, handling, and use characteristics of each grade. Polyacrylic acid polymers cross-linked with divinyl glycol are available for bioadhesive or medicinal applications. Carbomers designated with the letter ‘P’, for example, 971P, which may be used in the present invention, are the only pharmaceutical grades of polymer accepted for oral or mucosal contact products.

The mucoadhesive agent in the pharmaceutical composition helps the formulation to bind to the gastrointestinal mucosal lining. It also helps in the retention of the formulation in the upper parts of the gastrointestinal tract for longer periods of time. Once administered, the pharmaceutical composition of the present invention undergoes disintegration, after floating for a sufficient period of time. The disintegrated particles adhere to the mucosa of the stomach lining where the secretion of the acid will be maximal. The formulation will be retained in the stomach for longer periods of time, because of floatation and mucoadhesion. This will ensure a substantially complete conversion of elemental iron to the absorbable ferrous form, or at least a 70 percent conversion. The amount of mucoadhesive agent in the composition generally varies from about 1% to about 20% by weight of the composition. Preferably, the amount of mucoadhesive agent varies from about 2% to about 15% by weight of the composition.

The pharmaceutical composition of the present invention may also contain other required pharmaceutically acceptable excipients. The other required pharmaceutically acceptable excipients used in the present invention may be glidants and lubricants that are typically used in the pharmaceutical arts for oral solid dosage forms. The lubricants of the present invention are selected from those lubricants typically used in the pharmaceutical art for oral solid dosage forms. The amount of lubricant generally varies from about 0.1% to about 5.0% by weight of the composition.

The pharmaceutical composition of the present invention may contain other optional ingredients that are also typically used in pharmaceuticals, for example, coloring agents, preservatives, and flavorings. The amount of optional ingredients generally varies from about 0.1% to about 5.0% by weight of the composition.

The pharmaceutical composition of the present invention may be optionally coated with a coating, using the polymers or other coating agents which may or may not be intended or designed for the modification of drug release. For example cellulose ethers, such as ethyl cellulose, hydroxypropyl methyl cellulose, hydroxypropyl cellulose; polyvinyl alcohol, polyvinyl pyrrolidone, methacrylic acid derivatives, resins, clays, long chain hydrocarbons, long chain carboxylic acids, long chain carboxylic acid esters, long chain alcohols or mixtures thereof. The coating composition may include the coating polymer and other required additives like plasticizers, opacifiers, colorants, and preservatives. The weight gain of the coating generally varies from about 0.1% to about 10.0% by weight of the composition.

The pharmaceutical formulation of the present invention may be in the form of a tablet. Carbonyl iron has a high density and tends to fall to the bottom of a typical tablet mixture made up of carbonyl iron and excipients. This creates a non-homogenous mixture. In addition, carbonyl iron has poor compressibility characteristics resulting in a lack of a propensity to form a tablet when compacted. To remedy this problem, a carrier is used. As discussed above, the carrier may be a directly compressible grade of sorbitol, such as Sorbitol Instant. It is well known that the instant combination of carbonyl iron and sorbitol is successfully tabletted. This results because of sorbitol's extremely high adsorption potential for carbonyl iron and its ability to retain adsorption without carbonyl iron segregating from the sorbitol carrier. This occurs despite the significant density differences between the sorbitol carrier and carbonyl iron.

The tablet prepared is highly uniform and pharmaceutically acceptable. The tablet may have a hardness in the order of 50 N to 250 N, and more preferably, 100 N to 200 N as determined by a Schleuniger hardness tester. The compressed tablets can be optionally coated with a coating, using the polymers or other coating agents which may or may not be intended or designed for the modification of drug release.

According to present invention, the pharmaceutical composition is prepared by first blending the drug and a portion of sorbitol to ensure uniform adsorption of the drug with the carrier. The viscolyzing agent, gas generating agent, swelling agent, mucoadhesive agent, and remaining sorbitol are mixed together and then blended with the drug adsorbed over sorbitol. The granules are lubricated and then compressed into tablets. The tablets may be coated with methacrylic acid copolymer based film coats in order to give the tablets a colored and shiny clear appearance.

The following examples are provided to enable one skilled in the art to practice the invention and are merely illustrative of the invention. The examples should not be read as limiting the scope of the claims.

### EXAMPLES

### Example 1

Carbonyl Iron was sifted through ASTM mesh no. 60 and was adsorbed over a portion of sorbitol (carrier) that was previously sifted through ASTM mesh no. 20. Cross-linked polyvinyl pyrrolidone (swelling agent), sodium carboxy methyl cellulose (auxiliary swelling agent), polyethylene oxide WSR N80 (viscolyzing agent), sodium bicarbonate (gas generating agent) and microcrystalline cellulose (diluent) were sifted together through ASTM mesh no. 40. The remaining sorbitol was sifted through ASTM mesh no. 20 and was added to the above mentioned polymers. It was then blended uniformly with the carbonyl iron adsorbed over sorbitol. The granules were lubricated with magnesium stearate and compressed into tablets.

TABLE 1-1

| | | | % w/w of |
|---|---|---|---|
| | | Qty./ | unit |
| Sr. | | unit | dosage |
| No. | Ingredient | (mg) | form |
| 1. | Carbonyl Iron | 45.00 | 6.43 |
| 2. | Sorbitol Instant | 210.00 | 30.00 |
| 3. | Sodium Carboxy | 15.00 | 2.15 |
| | Methylcellulose | | |
| 4. | Cross-linked Polyvinyl | 85.00 | 12.14 |
| | Pyrrolidone | | |
| 5. | Polyethylene Oxide WSR N80 | 50.00 | 7.15 |
| 6. | Carbopol 971P | 70.00 | 10.00 |
| 7. | Sodium Bicarbonate | 42.00 | 6.00 |
| 8. | Microcrystalline Cellulose | 106.00 | 15.14 |
| 9. | Sorbitol Instant | 70.00 | 10.00 |
| 10. | Magnesium stearate | 7.00 | 1.00 |

The drug release profile from the dosage form of the invention was studied in 900 ml of 0.1 N Hydrochloric Acid, to simulate the gastric vironment, in USP Dissolution Apparatus Type I, with 10 mesh basket, at 100 RPM at about 37° C. The results showed a controlled release of the drug from the dosage form of the example 1. The drug release from the dosage form mentioned in example 1 was extended for more than 4 hours, as shown in table 1-2.

TABLE 1-2

| | % Cumulative |
|---|---|
| Time (h) | Drug Released |
| 0 | 0 |
| 0.5 | 5 |
| 1 | 10 |
| 2 | 25 |
| 3 | 35 |
| 4 | 43 |

### Example 2

In this example, calcium carboxy methylcellulose was used instead of the sodium salt. The method or preparation of the tablets is similar to that mentioned in example 1.

TABLE 2-1

| | | Qty./ | % w/w of |
|---|---|---|---|
| Sr. | | unit | unit dosage |
| No. | Ingredient | (mg) | form |
| 1. | Carbonyl Iron | 45.00 | 6.43 |
| 2. | Sorbitol Instant | 210.00 | 30.00 |
| 3. | Calcium Carboxy | 50.00 | 7.14 |
| | Methylcellulose | | |
| 4. | Cross-linked Polyvinyl | 85.00 | 12.14 |
| | Pyrrolidone | | |
| 5. | Polyethylene Oxide WSR N80 | 15.00 | 2.15 |
| 6. | Carbopol 971P | 70.00 | 10.00 |
| 7. | Sodium Bicarbonate | 42.00 | 6.00 |
| 8. | Microcrystalline Cellulose | 106.00 | 15.14 |
| 9. | Sorbitol Instant | 70.00 | 10.00 |
| 10. | Magnesium stearate | 7.00 | 1.00 |

The drug release profile from the dosage form of the invention was studied in 900 ml of 0.1 N Hydrochloric Acid, to simulate the gastric vironment, in USP Dissolution Apparatus Type I, with 10 mesh basket, at 100 RPM at about 37° C. The results showed a controlled release of the drug from the dosage form of the example 2. The drug release from the dosage form mentioned in example 2 was extended for more than 4 hours as shown in table 2-2.

TABLE 2-2

| | % Cumulative |
|---|---|
| Time (h) | Drug Released |
| 0 | 0 |
| 0.5 | 5 |
| 1 | 20 |
| 2 | 45 |
| 3 | 58 |
| 4 | 65 |

### Example 3

Tablets are prepared using the formula composition mentioned in table 3-1 and using the process similar to the one mentioned in example 1.

TABLE 3-1

| | | | % w/w of |
|---|---|---|---|
| | | Qty./ | unit |
| Sr. | | unit | dosage |
| No. | Ingredient | (mg) | form |
| 1. | Carbonyl Iron | 45.00 | 6.43 |
| 2. | Sorbitol Instant | 210.00 | 30.00 |
| 3. | Calcium Carboxy | 40.00 | 5.17 |
| | Methylcellulose | | |
| 4. | Cross-linked Polyvinyl | 75.00 | 10.71 |
| | Pyrrolidone | | |
| 5. | Polyethylene Oxide WSR N80 | 15.00 | 2.14 |
| 6. | Carbopol 971P | 60.00 | 8.57 |
| 7. | Sodium Bicarbonate | 42.00 | 6.00 |
| 8. | Sorbitol Instant | 206.00 | 29.42 |
| 9. | Magnesium stearate | 7.00 | 1.00 |

The drug release profile from the dosage form of the invention was studied in 900 ml of 0.1 N Hydrochloric Acid, to simulate the gastric vironment, in USP Dissolution Apparatus type I, with 10 mesh basket, at 100 RPM at about 37° C. The results showed a controlled release of the drug from the dosage form of the example 3. The drug release from the dosage form mentioned in example 3 was extended for more than 4 hours as shown in table 3-2.

TABLE 3-2

| | % Cumulative |
|---|---|
| Time (h) | Drug Released |
| 0 | 0 |
| 0.5 | 7 |
| 1 | 17 |
| 2 | 36 |
| 3 | 47 |
| 4 | 51 |

### Example 4

Tablets are prepared using the formula composition mentioned in table 4-1 and using the process similar to the one mentioned in example 1.

TABLE 4-1

| | | Qty./ | % w/w of |
|---|---|---|---|
| Sr. | | unit | unit dosage |
| No. | Ingredient | (mg) | form |
| 1. | Carbonyl Iron | 45.00 | 6.43 |
| 2. | Sorbitol Instant | 210.00 | 30.00 |
| 3. | Cross-linked Polyvinyl | 65.00 | 9.29 |
| | Pyrrolidone | | |
| 4. | Polyethylene Oxide WSR N80 | 15.00 | 2.14 |
| 5. | Carbopol 971P | 40.00 | 5.71 |
| 6. | Sodium Bicarbonate | 7.00 | 1.00 |
| 7. | Sorbitol Instant | 311.00 | 44.43 |
| 8. | Magnesium Stearate | 7.00 | 1.00 |

The drug release profile from the dosage form of the invention was studied in 900 ml of 0.1 N Hydrochloric Acid, to simulate the gastric environment, in USP Dissolution Apparatus Type I, with 10 mesh basket, at 100 RPM at about 37° C. The results showed a controlled release of the drug from the dosage form of the example 4. The drug release from the dosage form mentioned in example 4 was extended for more than 4 hours as shown in table 4-2.

TABLE 4-2

| | % Cumulative |
|---|---|
| Time (h) | Drug Released |
| 0 | 0 |
| 0.5 | 5 |
| 1 | 20 |
| 2 | 47 |
| 3 | 62 |
| 4 | 67 |

### Example 5

Tablets are prepared using the formula composition mentioned in table 5-1 and using the process similar to the one mentioned in example 1. The tablets are coated with Eudragit® E 100, a methacrylic acid copolymer available from BASF® of Ludwigshafen, Germany, to achieve a weight gain of about 2% and to give the tablets a colored, shiny and clear appearance.

TABLE 5-1

| | | Qty./ | % w/w of |
|---|---|---|---|
| Sr. | | unit | unit dosage |
| No. | Ingredient | (mg) | form |
| 1. | Carbonyl Iron | 45.00 | 6.43 |
| 2. | Sorbitol Instant | 210.00 | 30.00 |
| 3. | Cross-linked Polyvinyl | 75.00 | 10.71 |
| | Pyrrolidone | | |
| 4. | Polyethylene Oxide WSR N80 | 10.00 | 1.43 |
| 5. | Carbopol 971P | 40.00 | 5.71 |
| 6. | Sodium Bicarbonate | 7.00 | 1.00 |
| 7. | Sorbitol Instant | 306.00 | 43.71 |
| 8. | Magnesium Stearate | 7.00 | 1.00 |

The drug release profile from the dosage form of the invention was studied in 900 ml of 0.1 N Hydrochloric Acid, to simulate the gastric environment, in USP Dissolution Apparatus Type I, with 10 mesh basket, at 100 RPM at about 37° C. The results showed a controlled release of the drug from the dosage form of the example 5. The drug release from the dosage form mentioned in example 5 was extended for more than 4 hours as shown in table 5-2.

TABLE 5-2

| | % Cumulative |
|---|---|
| Time (h) | Drug Released |
| 0 | 0 |
| 0.5 | 14 |
| 1 | 29 |
| 2 | 49 |
| 3 | 61 |
| 4 | 70 |

### Example 6

Tablets are prepared using the formula compositions mentioned in table 6-1 and using the process similar to the one mentioned in example 1.

TABLE 6-1

| | | | % w/w of |
|---|---|---|---|
| | | Qty./ | unit |
| Sr. | | unit | dosage |
| No. | Ingredient | (mg) | form |
| 1. | Carbonyl Iron | 45.00 | 6.43 |
| 2. | Sorbitol Instant | 210.00 | 30.00 |
| 3. | Cross-linked Polyvinyl | 75.00 | 10.71 |
| | Pyrrolidone | | |
| 4. | Polyethylene Oxide WSR N80 | 15.00 | 2.14 |
| 5. | Carbopol 971P | 30.00 | 4.28 |
| 6. | Sodium Bicarbonate | 7.00 | 1.00 |
| 7. | Sorbitol Instant | 311.00 | 44.43 |
| 8. | Magnesium Stearate | 7.00 | 1.00 |

The drug release profile from the dosage form of the invention was studied in 900 ml of 0.15 N Hydrochloric Acid, to simulate the pH and the ionic content of the gastric environment where the tablet will be located after administration, in USP Dissolution Apparatus Type I, with 10 mesh basket, at about 37° C. The results showed a controlled release of the drug from the dosage form of the example 6. The drug release from the dosage form mentioned in example 6 for more than 4 hours as shown in table 6-2.

TABLE 6-2

| | % Cumulative |
|---|---|
| Time (h) | Drug Released |
| 0 | 0 |
| 0.5 | 3 |
| 1 | 16 |
| 2 | 43 |
| 3 | 59 |
| 4 | 67 |

### Example 7

Tablets are prepared using the formula composition mentioned in table 7-1 and using the process similar to the one mentioned in example 1.

TABLE 7-1

| | | Qty./ | % w/w of |
|---|---|---|---|
| Sr. | | unit | unit dosage |
| No. | Ingredient | (mg) | form |
| 1. | Carbonyl Iron | 45.000 | 6.430 |
| 2. | Folic Acid | 1.500 | 0.214 |
| 3. | Vitamin B 12 | 0.015 | 0.105 |
| 4. | Sorbitol Instant | 210.000 | 30.000 |
| 5. | Cross-linked Polyvinyl | 75.000 | 10.710 |
| | Pyrrolidone | | |
| 6. | Polyethylene Oxide WSR N80 | 15.000 | 2.140 |
| 7. | Carbopol 971P | 30.000 | 4.280 |
| 8. | Sodium Bicarbonate | 7.000 | 1.000 |
| 9. | Sorbitol Instant | 309.485 | 44.212 |
| .01 | Magnesium Stearate | 7.000 | 1.000 |

The drug release profile of Carbonyl Iron from the dosage form of the invention was studied in 900 ml of 0.15 N Hydrochloric Acid, to simulate the pH and the ionic content of the gastric environment where the tablet will be located after administration, in USP Dissolution Apparatus Type I, with 10 mesh baskets, at 100 RPM at about 37° C. The results showed a controlled release of the drug from the dosage form of the example 7. The drug release from the dosage form mentioned in example 7 was extended for more than 4 hours as shown in table 7-2.

TABLE 7-2

| | % Cumulative |
|---|---|
| Time (h) | Drug Released |
| 0 | 0 |
| 0.5 | 4 |
| 1 | 17 |
| 2 | 45 |
| 3 | 58 |
| 4 | 70 |

The drug release profiles of Examples 1 through 7 reveal that the dosage forms of the present invention extend the release of carbonyl iron for a period in excess of about 4 hours in the gastric environment. This extended release profile of the drug will contribute to the enhanced absorption of the ferrous form of iron form the dosage form.

Experiments to Determine the Conversion of Carbonyl, Iron to Ferrous and Ferric Form:

The ferrous salt form is three times more readily soluble than the ferric form, so experiments were performed to determine the rate of conversion of elemental iron into its most absorbable ferrous form. The dissolution conditions used were as follows.

Apparatus: USP Type II, 100 RPM Dissolution

Medium: 0.1N HCl, 900 ml

Temperature: 37° C.

Method of analysis for Total Iron: AAS

Rate of conversion experiment: Using Titrimetry

The drug release profile was studied for a period of about 4 hours. The samples were diluted and analyzed by titrimetry against known standards. Drug release studies were performed in vitro on the carbonyl iron modified release tablets of the present invention and marketed preparations like Feosol® caplets, Fefol Z spansules, both available from GlaxoSmithKline® of London, UK. The conditions used for the studies were as follows.

The studies revealed that the iron released in the pharmaceutical composition of the present invention is of the ferrous form when compared with the pure elemental iron and with the marketed preparation.

TABLE 8

Time (in minutes)

| | | | | | | |
|---|---|---|---|---|---|---|
| FORMULATION | 15 | 30 | 45 | 60 | 120 | 240 |

Carbonyl Iron API

| | | | | | | |
|---|---|---|---|---|---|---|
| Ferrous content | 53 | 80 | 85 | 83 | — | — |
| Total Iron | 60 | 90 | 93 | 94 | — | — |
| content | | | | | | |
| % of Ferrous Form | 88 | 89 | 91 | 88 | — | — |

Dosage Form of the Invention

| | | | | | | |
|---|---|---|---|---|---|---|
| Ferrous content | 9 | 26 | 36 | 49 | 69 | 74 |
| Total Iron | 11 | 27 | 41 | 54 | 76 | 78 |
| content | | | | | | |
| % of ferrous form | 82 | 96 | 87 | 91 | 91 | 95 |

Marketed Product: Feosol Caplets

| | | | | | | |
|---|---|---|---|---|---|---|
| Ferrous content | 19 | 46 | 64 | 74 | — | — |
| Total Iron | 27 | 59 | 78 | 88 | — | — |
| content | | | | | | |
| % of ferrous form | 70 | 78 | 82 | 84 | — | — |

1. A carbonyl iron pharmaceutical composition comprising:
(a) from about 1 percent weight to about 15 percent weight of carbonyl iron;
(b) from about 10 percent weight to about 90 percent weight of a carrier;
(c) from about 1 percent weight to about 25 percent weight of a swelling agent;
(d) from about 0.1 percent weight to about 20 percent weight of a viscolyzing agent;
(e) from about 0.1 percent weight to about 20 percent weight of a gas generating agent; and
(f) from about 1 percent weight to about 20 percent weight of a mucoadhesive agent.

2. The composition of claim 1, wherein the dosage form is a tablet, caplet or capsule.

3. The composition of claim 1, wherein at least 70 percent of said carbonyl iron is converted to the absorbable ferrous form.

4. The composition of claim 1, wherein said composition further comprises a lubricant.

5. The composition of claim 4, wherein said lubricant comprises from about 0.1 percent weight to about 5 percent weight of the composition.

6. The composition of claim 1, wherein said composition further comprises a coating layer.

7. The composition of claim 6, wherein said coating layer comprises from about 0.1 percent weight to about 10 percent weight of the composition.

8. The composition of claim 1, wherein said composition further comprises Vitamin B12.

9. The composition of claim 1, wherein said composition further comprises Folic Acid.

10. The composition of claim 1, wherein said carrier is sorbitol.

11. The composition of claim 1, wherein said viscolyzing agent is polyethylene oxide.

12. The composition of claim 1, wherein said swelling agent is selected from the group consisting of sodium carboxymethylcellulose, calcium carboxymethylcellulose, cross-linked polyvinyl pyrrolidone, cross-linked carboxymethylcellulose, and sodium starch glycolate.

13. The composition of claim 1, wherein said mucoadhesive agent is selected from the group consisting of cross-linked polyacrylic acids and carbomers.

14. The composition of claim 1, wherein said gas generating agent is selected from the group consisting of carbonates, bicarbonates, and sulfites.

15. The composition of claim 1, wherein said carbonyl iron comprises from about 2 percent weight to about 10 percent weight of the composition.

16. The composition of claim 1, wherein said swelling agent comprises from about 5 percent weight to about 15 percent weight of the composition.

17. The composition of claim 1, wherein said viscolyzing agent comprises from about 0.25 percent weight to about 15 percent weight of the composition.

18. The composition of claim 1, wherein said gas generating agent comprises from about 0.25 percent weight to about 10 percent weight of the composition.

19. The composition of claim 1, wherein said mucoadhesive agent comprises from about 2 percent weight to about 15 percent weight of the composition.

20. A method for the treatment of iron deficiency anemia which comprises administering to a patient in need thereof an effective amount of a composition according to claim 1.

21. A method of making a carbonyl iron pharmaceutical composition comprising the steps of:
(a) blending carbonyl iron and a carrier;
(b) mixing a viscolyzing agent, a gas generating agent, a swelling agent, and a mucoadhesive agent;
(c) blending and granulating the mixture of step (a) with the mixture of step (b);
(d) forming the mixture of step (c) into a pharmaceutically acceptable dosage form.

22. The method of claim 21, wherein the method further comprises the step of coating said pharmaceutically acceptable dosage form.

23. A method for the treatment of iron deficiency anemia which comprises administering to a patient in need thereof an effective amount of a composition according to claim 22.

24. The method of claim 21, wherein said carrier is sorbitol.

25. The method of claim 21, wherein said viscolyzing agent is polyethylene oxide,

26. The method of claim 21, wherein said swelling agent is selected from the group consisting of sodium carboxymethylcellulose, calcium carboxymethylcellulose, cross-linked polyvinyl pyrrolidone, cross-linked carboxymethylcellulose, and sodium starch glycolate.

27. The method of claim 21, wherein said mucoadhesive agent is selected from the group consisting of cross-linked polyacrylic acids and carbomers.

28. The method of claim 21, wherein said gas generating agent is selected from the group consisting of carbonates, bicarbonates, and sulfites.

29. The method of claim 21, wherein said pharmaceutically acceptable dosage form is a tablet, caplet or capsule

30. The method of claim 21, wherein said carbonyl iron comprises between about 1 percent weight and about 15 percent weight of the composition.

31. The method of claim 30, wherein said carbonyl iron comprises between about 2 percent weight and about 10 percent weight of the composition.

32. The method of claim 21, wherein said swelling agent comprises between about 1 percent weight and about 25 percent weight of the composition.

33. The method of claim 32, wherein said swelling agent comprises between about 5 percent weight and about 15 percent weight of the composition.

34. The method of claim 21, wherein said viscolyzing agent comprises between about 0.1 percent weight and about 20 percent weight of the composition.

35. The method of claim 34, wherein said viscolyzing agent comprises between about 0.25 percent weight and about 15 percent weight of the composition.

36. The method of claim 21, wherein said gas generating agent comprises between about 0.1 percent weight and about 20 percent weight of the composition.

37. The method of claim 36, wherein said gas generating agent comprises between about 0.25 percent weight and about 10 percent weight of the composition.

38. The method of claim 21, wherein said mucoadhesive agent comprises between about 1 percent weight and about 20 percent weight of the composition.

39. The method of claim 38, wherein said mucoadhesive agent comprises between about 2 percent weight and about 15 percent weight of the composition.
